(19) United States
(12) Patent Application Publication
(10) Pub. No.: US 2020/0368353 A1
(43) Pub. Date: Nov. 26, 2020

(54) SYSTEMS AND METHODS FOR TOPICAL CREAMS FOR WARMING

(71) Applicant: Innovus Pharmaceuticals, Inc., San Diego, CA (US)

(72) Inventors: Stephen R. Gladstone, Lincoln, MA (US); William Swartz, Delray Beach, FL (US)

(73) Assignee: Innovus Pharmaceuticals, Inc., San Diego, CA (US)

(21) Appl. No.: 16/762,261

(22) PCT Filed: Nov. 26, 2018
(86) PCT No.: PCT/US2018/062440

§ 371 Date: May 7, 2020

Related U.S. Application Data
(60) Provisional application No. 62/590,756, filed on Nov. 27, 2017.

(51) Int. Cl. A61K 41/00; A61K 31/198; A61K 31/165; A61K 47/02; A61K 31/085; A61K 9/06; A61K 9/00; A61K 47/44; A61K 47/14; A61K 47/10

(52) U.S. Cl. CPC A61K 41/0052; A61K 31/198; A61K 31/165; A61K 47/02; A61K 47/10; A61K 9/06; A61K 9/0014; A61K 47/44; A61K 47/14; A61K 31/085

(57) ABSTRACT

The present invention generally relates to the transdermal delivery of substances, e.g., for warming skin. Some aspects of the invention are generally directed to formulations that are able to deliver warming sensations to the skin to the skin within seconds, as opposed to minutes as in certain prior art formulations. In some embodiments, the composition may include warming agents, such as vanillyl butyl ether, which have an onset time of less than 1 minute, in addition to more longer-term warming agents such as oleoresin of capsicum. Other components, such as menthoxypropanediol, may be used in some cases, e.g., to provide a cooling sensation in addition to the warming effects of the warming agents. Surprisingly, this may enhance the warming effect of the warming agent. In addition, in some cases, the formulation may contain a nitric oxide donor, such as L-arginine, which may increase blood flow in the skin, and/O or a high salt environment to facilitate delivery into the skin.

### RELATED APPLICATIONS

This application claims the benefit of U.S. Provisional Patent Application Ser. No. 62/590,756, filed Nov. 27, 2017, entitled “Systems and Methods for Topical Creams for Warming,” by Gladstone, et al., incorporated herein by reference in its entirety.

### FIELD

The present invention generally relates to the transdermal delivery of substances, e.g., for warming skin.

### BACKGROUND

A variety of methods have been used to improve local blood flow within the skin, e.g., to warm the skin. These include a variety of surface treatments, including creams, gels, liquids, lotions, sprays, aerosols, transdermal patches, or the like. However, the onset of relief from such surface treatments may be slow, e.g., taking tens of minutes or hours before significant warming is perceived by the subject. Accordingly, improvements to such treatments are needed.

### SUMMARY

The present invention generally relates to the transdermal delivery of substances, e.g., for warming skin. The subject matter of the present invention involves, in some cases, interrelated products, alternative solutions to a particular problem, and/or a plurality of different uses of one or more systems and/or articles.

In one aspect, the present invention is generally directed to a topical delivery vehicle for application to the skin of a subject. In one set of embodiments, the topical delivery vehicle comprises L-arginine and/or an L-arginine salt, one or more ionic salts at a concentration of at least 5 wt %, oleoresin of capsicum, a warming agent having an onset time of less than 1 minute, and a cooling agent. In some cases, the topical delivery vehicle is a cream.

The topical delivery vehicle, in another set of embodiment, comprises a nitric oxide donor, one or more capsaicin compounds, a warming agent having an onset time of less than 1 minute, and a cooling agent. In certain instances, the topical delivery vehicle has an ionic strength of at least 0.25 M. In some cases, the topical delivery vehicle is a cream, gel, lotion, or ointment.

According to yet another set of embodiments, at least about 80% by weight of the topical delivery vehicle comprises water, sodium chloride and/or potassium chloride, L-arginine and/or L-arginine HCl, oleoresin of capsicum, vanillyl butyl ether, menthoxypropandiol, and an oil.

In still another set of embodiments, the topical delivery vehicle is a cream. In some cases, the topical delivery vehicle comprises water, one or more ionic salts, one or more nitric oxide donors, oleoresin of capsicum, vanillyl butyl ether, menthoxypropandiol, an oil, one or more triglycerides, phenoxyethanol, one or more stearates, cetyl alcohol, and methylparaben and/or propylparaben.

In another set of embodiments, the topical delivery vehicle consists essentially of: water, one or more ionic salts, one or more nitric oxide donors, oleoresin of capsicum, vanillyl butyl ether, menthoxypropandiol, an oil, one or more triglycerides, phenoxyethanol, one or more stearates, cetyl alcohol, and methylparaben and/or propylparaben.

In yet another set of embodiments, the topical delivery vehicle comprises each of the following compounds at concentrations that differ no more than +/−20% of the stated concentrations: water at a concentration of 30% to 75% by weight, sodium chloride and/or potassium chloride at a concentration of 5% to 15% by weight, L-arginine and/or L-arginine HCl at a concentration of 5% to 15% by weight, oleoresin of capsicum at a concentration of 0.1% to 5% by weight, vanillyl butyl ether at a concentration of 0.1% to 5% by weight, menthoxypropandiol at a concentration of 0.1% to 5% by weight, mineral oil at a concentration of 5% to 20% by weight, one or more triglycerides at a concentration of 1% to 10% by weight, one or more stearates at a concentration of 1% to 10% by weight, phenoxyethanol at a concentration of 0.1% to 5% by weight, and methylparaben and/or propylparaben at a concentration of 0.1% to 5% by weight.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

The present invention, in another aspect, is directed to a method of making one or more of the embodiments described herein. In yet another aspect, the present invention is directed to a method of using one or more of the embodiments described herein. In still another aspect, the present invention is directed to a method of promoting one or more of the embodiments described herein.

Other advantages and novel features of the present invention will become apparent from the following detailed description of various non-limiting embodiments of the invention.

### DETAILED DESCRIPTION

The present invention generally relates to the transdermal delivery of substances, e.g., for warming skin. Some aspects of the invention are generally directed to formulations that are able to deliver warming sensations to the skin to the skin within seconds, as opposed to minutes as in certain prior art formulations. In some embodiments, the composition may include warming agents, such as vanillyl butyl ether, which have an onset time of less than 1 minute, in addition to more longer-term warming agents such as oleoresin of capsicum. Other components, such as menthoxypropanediol, may be used in some cases, e.g., to provide a cooling sensation in addition to the warming effects of the warming agents. Surprisingly, this may enhance the warming effect of the warming agent. In addition, in some cases, the formulation may contain a nitric oxide donor, such as L-arginine, which may increase blood flow in the skin, and/or a high salt environment to facilitate delivery into the skin.

Certain aspects provide compositions for the topical delivery of substances to warm and/or cool the skin, including those discussed herein. The substances may be applied to the skin of a subject, e.g. a human, for example, to treat various symptoms (for instance, itch, pain, or the like). Such administration may be systemic or localized, e.g., directed to a specific location of the body of a subject. For example, a composition may be applied to locations such as the head, an arm, a hand, a leg, a foot, a thigh, the torso, the back, the neck, etc., depending on the specific application. In some cases, a composition is applied to a location where heating and/or cooling is desired.

In one set of embodiments, the composition comprises a warming agent having an onset time of less than about a minute, in addition to components that are able to deliver more long-term warming, such as discussed herein, and/or nitric oxide donors such as L-arginine and/or L-arginine salts. However, because such longer-term effects may take several minutes to hours to have an effect. For example, some nitric oxide donors need to be converted to nitric oxide, which then act to dilate blood vessels to the skin, thereby increasing blood flow and produce a warming sensation, which can often take at least 20 minutes to have an effect. In contrast, certain embodiments of the invention include additional agents that are able to cause a warming sensation more rapidly in the skin, for example, in less than 5 minutes, less than 4 minutes, less than 3 minutes, less than 2 minutes, or less than 1 minute. Such agents may also cause the user to perceive warming more quickly, e.g., within 5 minutes or within a minute, in contrast to compositions that do not contain such agents.

One example of a warming agent having such rapid onset times is vanillyl butyl ether (4-(butoxymethyl)-2-methoxy-phenol). Other examples include, but are not limited to menthol, methone, vitamin E nicotinate, tocopherol nicotinate, cinnamomum camphora linalooliferum leaf oil, zeolite powder, peppermint oil, spearmint oil, cassia extract, etc. In addition, more than one such agent may be present within the composition, including any one or more of these and/or other such agents.

The warming agent may be present in any suitable concentration. For instance, the agent may be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.2%, at least about 2.5%, at least about 3%, at least about 3.5%, at least about 4%, at least about 4.5%, or at least about 5%. In some cases, the agent may be present at no more than about 5%, no more than about 4.5%, no more than about 4%, no more than about 3.5%, no more than about 3%, no more than about 2.5%, no more than about 2.2%, no more than about 2%, no more than about 1.9%, no more than about 1.8%, no more than about 1.7%, no more than about 1.6%, no more than about 1.5%, no more than about 1.4%, no more than about 1.3%, no more than about 1.2%, no more than about 1.1%, no more than about 1%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%. (All percentages are by weight.) Combinations of any of these are also possible. For example, a warming agent such as vanillyl butyl ether may be present at between 0.5% and 1.5% within a formulation.

As mentioned, in some embodiments, the composition may also contain one or more warming agents that have more longer-term effects, such as oleoresin of capsicum, or nitric oxide donors such as L-arginine and/or L-arginine salts. Examples of nitric oxide donors are discussed in more detail below. In some embodiments, such compounds may begin to have an effect within a few minutes. Thus, in combination with agents such as those described above, short- to long-term warming effects can be achieved in certain embodiments of the invention.

For instance, in one set of embodiments, the composition may contain one or more capsaicin compounds, such as capsaicin, dihydrocapsaicin, nordihydrocapsaicin, homocapsaicin, or the like. In some cases, the composition comprises oleoresin of capsicum (or oleoresin capsicum). Without wishing to be bound by any theory, it is believed that such capsaicin compounds are able to react with nerve endings within the skin, e.g., sensory nerves that sense heat or pain. Accordingly, in some cases, such compounds may be applied to the skin to cause a warming sensation.

Such warming agents may be present in any suitable concentration, and the concentrations may be the same or different as other warming agents, such as those previously discussed. For instance, the warming agent may be present at at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.2%, at least about 2.5%, at least about 3%, at least about 3.5%, at least about 4%, at least about 4.5%, or at least about 5% by weight. In some embodiments, the warming agent may be present at no more than about 5%, no more than about 4.5%, no more than about 4%, no more than about 3.5%, no more than about 3%, no more than about 2.5%, no more than about 2.2%, no more than about 2%, no more than about 1.9%, no more than about 1.8%, no more than about 1.7%, no more than about 1.6%, no more than about 1.5%, no more than about 1.4%, no more than about 1.3%, no more than about 1.2%, no more than about 1.1%, no more than about 1%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1% by weight. Combinations of any of these are also possible. For example, a warming agent such as oleoresin capsicum may be present at between 0.5% and 1.5% within a formulation.

In addition, in some embodiments, the composition may also contain one or more cooling agents. Without wishing to be bound by any theory, it is believed that the use of one or more cooling agents may be used to counter or enhance the effects of the warming agents. For example, it has been found that surprisingly, due to the paradoxical sensations of simultaneous cooling and warming, the subject may perceive an enhanced warming effect due to the warming agents applied to the skin.

One example of a cooling agent is menthoxypropandiol. Other examples of cooling agents that can be used include, but are not limited to, 2-isopropyl-N,2,3-trimethylbutyramide, N-ethyl-p-menthane-3-carboxamide, ethyl 3-(p-menthane-3-carboxamido)acetate, (1R,2S,5R)-N-(4-methoxyphenyl)-p-menthanecarboxamide, N-ethyl-2,2-diisopropylbutanamide, N-cyclopropyl-5-methyl-2-isopropylcyclohexanecarboxamide, N-(1,1-dimethyl-2-hydroxyethyl)-2,2-diethylbutanamide, N-(4-cyanomethylphenyl)-p-menthanecarboxamide, N-(2-(pyridin-2-yl)ethyl)-3-p-menthanecarboxamide, N-(2-hydroxyethyl)-2-isopropyl-2,3-dimethylbutanamide, or N-(4-(carbamoylmethyl)phenyl)-menthylcarboxamide.

The cooling agent may be present in any suitable concentration. For instance, the agent may be present at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.2%, at least about 2.5%, at least about 3%, at least about 3.5%, at least about 4%, at least about 4.5%, or at least about 5%. In some cases, the agent may be present at no more than about 5%, no more than about 4.5%, no more than about 4%, no more than about 3.5%, no more than about 3%, no more than about 2.5%, no more than about 2.2%, no more than about 2%, no more than about 1.9%, no more than about 1.8%, no more than about 1.7%, no more than about 1.6%, no more than about 1.5%, no more than about 1.4%, no more than about 1.3%, no more than about 1.2%, no more than about 1.1%, no more than about 1%, no more than about 0.9%, no more than about 0.8%, no more than about 0.7%, no more than about 0.6%, no more than about 0.5%, no more than about 0.4%, no more than about 0.3%, no more than about 0.2%, or no more than about 0.1%. (All percentages are by weight.) Combinations of any of these are also possible. For example, a cooling agent may be present at between 0.5% and 1.5% within a formulation.

In certain embodiments, the formulation comprises a first warming agent having an onset time of less than 1 minute, a second warming agent, and a cooling agent. For instance, in one set of embodiments, the first warming agent may include vanillyl butyl ether, the second warming agent may include oleoresin of capsicum and/or nitric oxide donors such as L-arginine and/or L-arginine salts, and the cooling agent may include menthoxypropandiol. Compared to high salt formulations for delivering L-arginine and/or L-arginine salts, such compositions may exhibit significantly improved onset times for warming and/or cooling.

In one set of embodiments, methoxypropandiol facilitates cooling sensations within the skin, and also increase the moisturizing and absorbance or penetration properties of the composition, e.g., to promote more rapid absorption of agents such as warming agents, especially warming agents having rapid onset times such as those discussed herein (e.g., vanillyl butyl ether). Methoxypropandiol also may improve the viscous properties of the composition, e.g., to reduce the amount of graininess or particle concentration with the composition, and to reduce the amount of residue left on the skin due to better absorption and viscosity of the composition within the skin.

Thus, according to certain embodiments, there is a synergy between the effects of methoxypropandiol in more rapidly increase absorbance of warming agents, and the ability of warming agents such as vanillyl butyl ether to warm the skin, relative to when one or both is not present.

As mentioned, the composition may comprise a nitric oxide donor in some embodiments. Examples include L-arginine and/or L-arginine salts (e.g., L-arginine hydrochloride). In some cases, such a nitric oxide donor may be used to increase localized blood flow at the site where the composition is applied. This may be useful, for example, for increasing warming and/or cooling at the site.

A nitric oxide donor, typically is a compound that is able to release nitric oxide and/or chemically transfer a nitric oxide moiety to another molecule, directly or indirectly, for example, through a biological process. The nitric oxide donor may release nitric oxide into the skin, and/or tissues such as muscles and/or elements of the circulatory system in close proximity to the surface of the skin. Non-limiting examples of nitric oxide donors include arginine (e.g., L-arginine and/or D-arginine), arginine salts (e.g., L-arginine hydrochloride and/or D-arginine hydrochloride, L-arginine citrate and/or D-arginine citrate, etc.), nitroglycerin, polysaccharide-bound nitric oxide-nucleophile adducts, N-nitroso-N-substituted hydroxylamines, 1,3-(nitrooxymethyl)phenyl-2-hydroxybenzoate, etc., and/or any combination of these and/or other compounds.

Other non-limiting examples of nitric oxide donors include D,L-arginine, D-arginine, or alkyl (e.g., ethyl, methyl, propyl, isopropyl, butyl, isobutyl, tert-butyl, etc.) esters of L-arginine and/or D-arginine (e.g., a methyl ester, an ethyl ester, a propyl ester, a butyl ester, etc.) and/or salts thereof, as well as other derivatives of arginine and other nitric oxide donors. For instance, non-limiting examples of pharmaceutically acceptable salts include hydrochloride, glutamate, butyrate, or glycolate (e.g., resulting in L-arginine glutamate, L-arginine butyrate, L-arginine glycolate, D-arginine hydrochloride, D-arginine glutamate, etc.). Still other examples of nitric oxide donors include L-arginine-based compounds such as, but not limited to, L-homoarginine, N-hydroxy-L-arginine, nitrosylated L-arginine, nitrosylated L-arginine, nitrosylated N-hydroxy-L-arginine, nitrosylated N-hydroxy-L-arginine, citrulline, ornithine, linsidomine, nipride, glutamine, etc., and salts thereof (e.g., hydrochloride, glutamate, butyrate, glycolate, etc.), and/or any combination of these and/or other compounds. Still other non-limiting examples of nitric oxide donors include S-nitrosothiols, nitrites, 2-hydroxy-2-nitrosohydrazines, or substrates of various forms of nitric oxide synthase. In some cases, the nitric oxide donor may be a compound that stimulates endogenous production of nitric oxide in vivo. Examples of such compounds include, but are not limited to, L-arginine, substrates of various forms of nitric oxide synthase, certain cytokines, adenosine, bradykinin, calreticulin, bisacodyl, phenolphthalein, OH-arginine, or endothelein, and/or any combination of these and/or other compounds.

The nitric oxide donor may be present at any suitable concentration within the composition. For instance, in some cases, the nitric oxide donor is present at a concentration of at least about 1%, at least about 2%, at least about 3%, at least about 4%, at least about 5%, at least about 6%, at least about 7%, at least about 7.5%, at least about 8%, at least about 9%, at least about 10%, at least about 11%, at least about 12%, at least about 13%, at least about 14%, or at least about 15% by weight of the composition. In some cases, the nitric oxide donor is present at a concentration of no more than about 15%, no more than about 14%, no more than about 13%, no more than about 12%, no more than about 11%, no more than about 10%, no more than about 9%, no more than about 8%, no more than about 7%, no more than about 6%, no more than about 5%, no more than about 4%, no more than about 3%, no more than about 2%, or no more than about 1% by weight of the composition. Combinations of any of these are also possible, in other embodiments; for example, a nitric oxide donor may be present at between about 10% and about 14% by weight, or between about 8% and about 10% by weight, etc.

As a particular non-limiting example, in certain embodiments, nitric oxide is provided using L-arginine, for example, at a concentration of at least about 0.5% by weight (wt % or w/v) of L-arginine, at least about 0.75 wt %, at least about 1 wt %, at least about 2 wt %, at least about 3 wt %, at least about 5 wt %, at least about 7 wt %, at least about 10 wt %, or at least about 15 wt %. The L-arginine may be present in a suitable delivery vehicle, such as a cream or a lotion. L-arginine and/or its salts (e.g., L-arginine HCl) may be particularly useful in some cases due to its low toxicity, its high solubility, and/or its low cost. Other examples of nitric oxide donors are discussed in Int. Pat. Apl. Pub. No. WO 2005/081964, incorporated herein by reference in its entirety.

Without wishing to be bound to any theory, it is generally believed that the flow of a substance across the skin may slow as it builds up within the tissue. Fick's first law of diffusion suggests that when the concentration inside becomes substantially equal to that outside, passive flow stops. The increased local blood flow may prevent or at least decrease the stoppage of the flow of the substance. Thus, when the composition is applied to the skin, the substance exits the vehicle into the tissue more readily, as the substance is dispersed by flow and does not build up in concentration in the tissue. Accordingly, the composition may be delivered locally and/or systemically; initially, much of the delivery is at first local (i.e., through the skin), but in some cases, the substance may also be distributed systemically, e.g., upon reaching the blood supply.

In some cases, one or more nitric oxide donors (e.g., 2, 3, 4, 5, 6, 7, 8, 9, 10, etc. nitric oxide donors) may be used. In some cases, there may be no more than 3, 5, 7, or 10 nitric oxide donors present within the composition.

The composition may comprise a hostile biophysical environment to the substance in some embodiments. In a hostile biophysical environment, the environment surrounding the substance may be such that the pharmaceutical agent is in a chemically and/or energetically unfavorable environment, relative to the skin (e.g., the chemical potential and/or the free energy of the pharmaceutical agent within the hostile biophysical environment is significantly greater than the chemical potential and/or the free energy of the substance within the skin, thus energetically favoring transport into the skin), especially the stratum corneum.

Non-limiting examples of such compositions are discussed in Int. Pat. Apl. Pub. No. WO 2005/102282, incorporated herein by reference. Other techniques for hostile biophysical environments are discussed in detail herein. Accordingly, certain embodiments of the invention are generally directed to compositions for topical delivery to the skin of a subject comprising a nitric oxide donor and a hostile biophysical environment.

A hostile biophysical environment of the invention can comprise, in one set of embodiments, a high ionic strength environment. For example, the composition may have an ionic strength of at least about 0.25 M, at least about 0.5 M, at least about 1 M, at least about 2 M, at least about 3 M, at least about 4 M, at least about 5 M, at least about 7 M, at least about 10 M, at least about 12 M, at least about 15 M, at least about 20 M, at least about 25 M, etc. In some cases, the ionic strength of the composition may be no more than about 25 M, no more than about 20 M, no more than about 15 M, no more than about 12 M, no more than about 10 M, no more than about 7 M, no more than about 5 M, no more than about 4 M, no more than about 3 M, no more than about 2 M, no more than about 1 M, no more than about 0.5 M, no more than about 0.25 M, etc. Combinations of any of these ionic strengths are also possible. For example, the ionic strength may be between about 0.25 M and about 15 M, between about 5 M and about 15 M, between about 10 M and about 15 M, etc. In some embodiments, the ionic strength is any amount greater than two times the physiological ionic strength of blood.

In some embodiments, the high ionic strength environment may be caused by the presence or one or more salts within the composition. For instance, the ionic strength of a composition can be readily controlled in certain embodiments by controlling the amounts or concentrations of one or more of the salts present in the composition. Examples of ionic salts that may be used include, but are not limited to, one or more of sodium chloride, magnesium chloride, potassium chloride, calcium chloride, choline chloride, lithium chloride, sodium bromide, magnesium bromide, potassium bromide, calcium bromide, choline bromide, lithium bromide, sodium iodide, magnesium iodide, potassium iodide, calcium iodide, choline iodide, lithium iodide, sodium citrate, magnesium citrate, potassium citrate, calcium citrate, choline citrate, lithium citrate, etc. Combinations of these and/or other salts may also be used in certain embodiments. In addition, other highly charged molecules such as polylysine, polyglutamine, polyaspartate, etc., or copolymers of such highly charged amino acids may also be used in certain embodiments.

In some cases, one or more salts may each independently be present at a concentration of at least about 0.25 M, at least about 0.5 M, at least about 1 M, at least about 2 M, at least about 3 M, at least about 4 M, at least about 5 M, at least about 7 M, at least about 10 M, at least about 12 M, at least about 15 M, at least about 20 M, at least about 25 M, etc. In some cases, the concentration may be no more than about 25 M, no more than about 20 M, no more than about 15 M, no more than about 12 M, no more than about 10 M, no more than about 7 M, no more than about 5 M, no more than about 4 M, no more than about 3 M, no more than about 2 M, no more than about 1 M, no more than about 0.5 M, no more than about 0.25 M, etc. Combinations of any of these concentrations are also possible. For example, the ionic strength may be between about 0.25 M and about 15 M, between about 5 M and about 15 M, between about 10 M and about 15 M, etc. In addition, it should be noted that if more than one salt is present, each contributing to the overall ionic strength, then the concentrations of each may be independent of each other. In some cases, the concentrations of each may be relatively low although the sum total contribution of each to ionic strength may be used to produce a high ionic strength environment.

Thus, according to some embodiments of the invention, compositions comprising a relatively high salt composition (e.g., high chloride content) are effective for topical delivery of certain compounds. In some embodiments, salt-enhanced delivery is particularly effective. In some cases, one or more salts may each be present within composition at at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% (by weight). In some embodiments, one or more salts may each independently be present at no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1%. Combinations of any of these ranges are also possible in certain embodiments. For example, a salt such as sodium chloride may be present at between 5 wt % and 15 wt % in the composition. In addition, in some cases, the total amount of salt present within the composition may be within any of these ranges. Thus, for example, one or more salts (e.g., sodium chloride and/or potassium chloride) may be present within the composition such that the total amount of salt is between 5 wt % and 15 wt %.

In some cases, the pH of the composition may be optimized to ionize the compound being delivered (e.g., at least about 80%, at least about 90%, at least about 95%, or about 99% or more) is ionized. It should be appreciated that depending on the pKa of the compound and the pH of the composition, the ionized form may be anionic or cationic (e.g., due to protonation). In some embodiments, a compound may contain several ionizable groups each having a different pKa. In some embodiments, it is sufficient for at least 1, 2, or 3 of the groups to be ionized for the salt-enhanced delivery to be effective. In certain embodiments, an ionizable group is sufficiently ionized if the pH of the composition is at least 1 pH unit, or at least 2 pH units (e.g., 1, 1-2, 2-3, or more pH units) below the pKa of the group and it is cationic (due to protonation) below its pKa. Similarly, in some embodiments, an ionizable group is sufficiently ionized if the pH of the composition is at least 1 pH unit, or at least 2 pH units (e.g., 1, 1-2, 2-3, or more pH units) above the pKa of the group and it is anionic (due to deprotonation) above its pKa. In some embodiments, the presence of magnesium chloride, for example at 0.1-5% by weight, can help stabilize compositions containing compounds with relatively high pKas (e.g., above 8.0, above 9.0, above 10.0 or higher). In some embodiments, the pH of a composition may be maintained using a buffer. However, the pH of compositions of the invention are stable without a buffer. In some embodiments, a desired pH can be established by titrating the mixture with an acid (e.g., HCl) or a base (e.g., NaOH). The pH of the resulting composition (e.g., when formulated as an emulsion as described herein) can be stable (e.g., sufficiently for the composition to be effective for transdermal delivery) for extended periods of time (e.g., weeks, months, or 1 or more years).

In other embodiments, other methods may be used to produce a hostile biophysical environment, e.g., in addition to or instead of a high ionic strength environment. For instance, in one set of embodiments, a hostile biophysical environment may be produced using a high concentration of osmotic agents such as ureas, sugars, or carbohydrates, a high pH environment (e.g., greater than about 7, greater than about 8, greater than about 9, greater than about 10, greater than about 11, greater than about 12, or greater than about 13), a low pH environment (less than about 5, less than about 4, less than about 3 or less than about 2), highly hydrophobic components, or highly hydrophilic components or other substances that cause an increase in the chemical potential and/or free energy of the pharmaceutical agent, or any combination of two or more of these and/or other compounds. A hydrophobic component may, in some embodiments, have an octanol-water partition coefficient of at least about 100, at least about 1000, at least about 10⁴, at least about 10⁵, or more in some cases. Similarly, a hydrophilic component may have an octanol-water partition coefficient of less than about 0.01, less than about 10⁻³, less than about 10⁻⁴, or less than about 10⁻⁵ in some cases. In some cases, high or low pH environments (e.g., by adding pharmaceutically acceptable acids or bases, for example, such that the pH is between about 3 and about 7, between about 3 and about 6, between about 3 and about 5, between about 4 and 8, between about 5 and about 8, between about 5 and 8.5, between about 7 and about 11, between about 8 and about 11, between about 9 and about 11, etc.); or highly hydrophobic environments (e.g., by decreasing water content and increasing lipid, oil and/or wax content of the environment) may be used to create a biophysical hostile environment.

In some cases, the composition defines the biophysical hostile environment. In certain cases, a substance may be packaged in such a way that it is carried into tissue and/or its charge is neutralized by derivitization and/or by forming a neutral salt. Non-limiting examples of delivery vehicles which would be carried into tissue includes liposomes or emulsions of collagen, collagen peptides or other components of skin or basement membrane. Non-limiting examples of neutralization of charge include delivery of the substance in the form or an ester or salt which is electronically neutral. In some embodiments, the hostile biophysical environment may include any two or more of these conditions. For instance, the hostile biophysical environment may include high ionic strength and a high pH or a low pH, a highly hydrophobic environment and a high pH or a low pH, a highly hydrophobic environment that includes liposomes, or the like.

A hostile biophysical environment may also be created in some embodiments by placing a substance that is relatively highly charged into a hydrophobic, oily environment such as in an oil-based cream or lotion containing little or no water. Absorption may further be aided in some cases by combining the use of hostile biophysical environments with the use of penetrating agents, as further described herein.

In some embodiments, the composition may include one or more penetrating agents, e.g., that are able to increase transport across the skin, relative to transport in the absence of the penetrating agent. For instance, transport of the nitric oxide donor, and/or other substances such as those described herein, may be enhanced using one or more penetration agents. Non-limiting examples of penetrating agents include, but are not limited to, cationic, anionic, or nonionic surfactants (e.g., sodium dodecyl sulfate, polyoxamers, etc.); fatty acids and alcohols (e.g., ethanol, oleic acid, lauric acid, liposomes, etc.); anticholinergic agents (e.g., benzilonium bromide, oxyphenonium bromide); alkanones (e.g., n-heptane); amides (e.g., urea, N,N-dimethyl-m-toluamide); fatty acid esters (e.g., n-butyrate); organic acids (e.g., citric acid); polyols (e.g., ethylene glycol, propylene glycol, glycerol); sulfoxides (e.g., dimethylsulfoxide); terpenes (e.g., cyclohexene); ureas; sugars; carbohydrates or other agents (e.g., azone or laurocapram). In certain embodiments, the penetrating agent includes a salt, e.g., as described herein. Additional examples of penetrating agents include certain molecules containing heterocyclic rings to which are attached hydrocarbon chains.

In one set of embodiments, the composition may be present as an emulsion. As known by those of ordinary skill in the art, an emulsion typically includes a first phase (e.g., a discontinuous phase) contained within a second fluid phase (e.g., a continuous phase). The substance may be present in either or both phases. In addition, other materials such as those described herein may be present in the same phase as the substance. In some embodiments, the emulsion may take the form of a cream or a lotion. For example, a substance may be contained within a hydrophobic, oily environment such as in an oil-based cream or lotion containing little or no water.

For example, a cream may include materials such as oils, triglycerides, stearates, fatty acids, fatty alcohols, squalenes, polysorbates, or the like. In some cases, such materials are hydrophobic, which can be emulsified with water or other aqueous phases, e.g., to produce an emulsion. In one embodiment, for example, the cream may include a saturated squalene. Examples of stearates include, but are not limited to, glyceryl stearate, propylene glycol stearate, steryl stearate, sorbitan stearate, sodium stearate, calcium stearate, magnesium steratae, glycol sterate, and the like. Non-limiting examples of oils include mineral oil, wheat germ oil, palm oil, nut oil, linseed oil, etc. Other materials may also be present within the composition, for example, buffers, preservatives, surfactants, etc.

In some cases, the cream may include one or more thickening agents, which may be used to increase viscosity. One example of a thickening agent is xanthan gum. Other examples include, but are not limited to, guar gum, starch, carrageenan, carob gum, dextrin, cellulose, methyl cellulose, ethyl cellulose, carboxymethyl cellulose, hydroxyethyl cellulose, hydroxypropyl cellulose, carboxymethyl starch, pullulan, sodium alginate, ammonium alginate, propylene glycol alginate ester, locust bean gum, gum arabic, gelatin, casein, polyvinyl alcohol, polyethylene oxide, polyethylene glycol, ethylene/propylene block polymers, polyacrylates such as sodium polyacrylate, polyvinyl pyrrolidone, etc.

In one set of embodiments, as a non-limiting example, a cream may include one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A and D, triethanolamine, methylparaben, aloe vera extract, imidazolidinyl urea, propylparaben, PND, and/or BHA. As another example, a cream may include one or more of water, sodium chloride, potassium chloride, L-arginine HCl, mineral oil, caprylic/capric triglycerides, phenoxyethanol, glycerol stearate, PEG 75 stearate, cetyl alcohol, methylparaben, and propylparaben. As another example, a cream may include one or more of water, sodium chloride, potassium chloride, L-arginine HCl, mineral oil, caprylic/capric triglycerides, phenoxyethanol, glycerol stearate, PEG 75 stearate, cetyl alcohol, vanillyl butyl ether, menthoxypropandiol, oleoresin of capsicum, methylparaben, and propylparaben. As yet another example, a cream may include one or more of water, sodium chloride, potassium chloride, L-arginine HCl, mineral oil, caprylic/capric triglycerides, phenoxyethanol, glycerol stearate, PEG 75 stearate, cetyl alcohol, vanillyl butyl ether, oleoresin of capsicum, menthoxypropandiol, methylparaben, and propylparaben.

The above compounds may be present within the composition in any suitable amounts. For example, one or more of the above compositions may be present at at least about 0.1%, at least about 0.2%, at least about 0.3%, at least about 0.5%, at least about 1%, at least about 2%, at least about 3%, at least about 5%, at least about 10%, etc. In some cases, the compositions may be present at no more than about 10%, no more than about 5%, no more than about 3%, no more than about 2%, no more than about 1%, no more than about 0.5%, no more than about 0.3%, no more than about 0.2%, or no more than about 0.1%. Combinations of any of these percentages are also possible. The actual concentration for a particular application can be determined by those of ordinary skill in the art using no more than routine experimentation, for example, by measuring the amount of transport of a compound as a function of concentration in vitro across cadaver skin or suitable animal models, skin grafts, synthetic model membranes, human models, or the like.

In certain embodiments, compositions such as those described herein may provide moisturizing properties. For example, a cream as discussed herein may provide moisturization as well as warming sensations to a subject. In addition, in some cases, certain compositions as described herein may provide a less grainy or salty feel.

In one set of embodiments, at least 50 wt %, at least 60 wt %, at least 70 wt %, at least 75 wt %, at least 80 wt %, at least 85 wt %, at least 90 wt %, or at least 95 wt % of the composition comprises water, one or more ionic salts (e.g., sodium chloride and/or potassium chloride), one or more nitric oxide donors (e.g., L-arginine and/or L-arginine HCl), one or more capsaicin compounds (e.g., oleoresin of capsicum), one or more warming agents (e.g., vanillyl butyl ether), one or more cooling agents (e.g., menthoxypropandiol), and one or more oils (e.g., mineral oil). The composition may also contain other components, such as one or more stearates (e.g., glycerol stearate, PEG 75 stearate, etc.), one or more triglycerdies (e.g., caprylic and/or capric triglycerides), cetyl alcohol, phenoxyethanol, methylparaben, and/or propylparaben. In some cases, the composition consists essentially of one or more of the above.

In another set of embodiments, the composition comprises one or more of water at a concentration of 30% to 75% by weight, sodium chloride and/or potassium chloride at a concentration of 5% to 15% by weight, L-arginine and/or L-arginine HCl at a concentration of 5% to 15% by weight, oleoresin of capsicum at a concentration of 0.1% to 5% by weight, vanillyl butyl ether at a concentration of 0.1% to 5% by weight, menthoxypropandiol at a concentration of 0.1% to 5% by weight, mineral oil at a concentration of 5% to 20% by weight, one or more triglycerides at a concentration of 1% to 10% by weight, one or more stearates at a concentration of 1% to 10% by weight, phenoxyethanol at a concentration of 0.1% to 5% by weight, and methylparaben and/or propylparaben at a concentration of 0.1% to 5% by weight.

In some embodiments, various aspects of the invention relate to methods and compositions for preparing and/or manufacturing formulations such as those discussed herein. In one set of embodiments, the present invention is generally directed to emulsions that contain one or more substances described herein for topical application. In some embodiments, certain embodiments of the invention are useful for preparing emulsions that contain one or more substances in a hostile biophysical environment. In some embodiments, the hostile biophysical environment is a high salt concentration (e.g., a high concentration of one or more salts), for example, as described herein. Methods and compositions such as any of those discussed herein may be used to prepare a composition that is sterile or that has a low microbial content, in some embodiments.

In some embodiments, an emulsion is prepared by mixing a first aqueous preparation (e.g., a water phase) with a second non-aqueous preparation (e.g., an oil or lipid phase). Substances that are water-soluble may be added to the first aqueous preparation (e.g., prior to mixing with the second non-aqueous preparation). Substances that are water insoluble (or relatively water insoluble) may be added to the second non-aqueous preparation (e.g., prior to mixing with the first aqueous preparation). Substances that are partially water soluble may be added to one phase, or may be split between the two phases prior to mixing. The split between the two phases will depend on factors such as the amount of the substance that is being added, the composition (e.g., the nature and the amount of other chemicals or agents) of the first and second preparations, the pH, the temperature, other physical or chemical factors, and/or a combination thereof. For example, if a substance is soluble at a 1% level in the aqueous (e.g., water or buffer) phase, but a 2% level of the drug is required in the emulsion, then the substance may also be added to the non-aqueous (e.g., lipid) phase at a 1% level. In some embodiments, a substance that is less than 1% soluble in an aqueous phase is provided in the non-aqueous phase prior to mixing. However, it should be appreciated that other percentages and/or splits between the two phases may be used.

In some embodiments, the pH of one or both of the first and second preparations is adjusted to optimize the solubility of the substance. In some embodiments, a high salt concentration is used. In order to prevent a high salt concentration from breaking down an emulsion, one or more emulsifying agents may be used in some cases. In some embodiments, the mixing time may be adjusted to promote appropriate mixing and/or emulsion formation.

In some embodiments, the temperature of the first and/or second preparation may be controlled to promote solubility, mixing, and/or emulsion formation. In some embodiments, the temperature of one or both preparations and/or of the mixing may be set at 25° C. or higher (e.g., 30° C. or higher, 40° C. or higher, 50° C. or higher, 60° C. or higher, 70° C. or higher, or 80° C. or higher). For example, the temperature may be at between 30° C. and 90° C., between 40° C. and 80° C., at around 50° C., at around 60° C., or at around 70° C.

Emulsions of the invention may be packaged using any suitable format (e.g., in a tube, a pump-actuated container, or any other suitable form), in certain embodiments of the invention. For example, in some embodiments, an emulsion may be added to a surface of a patch or bandage. The emulsion may also be applied to the skin of a subject as a cream, gel, liquid, lotion, spray, aerosol, or the like.

In some aspects, a composition as discussed herein is administered to a subject. Such administration may be systemic or localized, e.g., directed to a specific location of the body of a subject. The composition may be applied in any suitable form, e.g., as discussed herein. For example, the composition may be applied using a delivery vehicle such as a cream, gel, liquid, lotion, spray, aerosol, or transdermal patch. In one set of embodiments, a composition may be applied or impregnated in a bandage or a patch applied to the skin of a subject. In some embodiments, a patch has a skin contacting portion made of any suitable material that is covered or impregnated with a cream or emulsion described herein, wherein the skin contacting portion may be supported by a backing, one or both of which may have an adhesive segment or other configuration for attaching to the skin surface of a subject.

A subject typically includes humans and/or non-human animals. Examples of subjects include, but are not limited to, a mammal such as a dog, a cat, a horse, a donkey, a rabbit, a cow, a pig, a sheep, a goat, a rat (e.g., Rattus norvegicus), a mouse (e.g., Mus musculus), a guinea pig, a hamster, a primate (e.g., a monkey, a chimpanzee, a baboon, an ape, a gorilla, etc.), or the like. Such delivery vehicles may be applied to the skin of a subject, such as a human subject. Examples of delivery vehicles are discussed herein. The delivery vehicle may promote transfer into the skin of an effective concentration of the nitric oxide donor and/or the pharmaceutical agent, directly or indirectly. For instance, the delivery vehicle may include one or more penetrating agents, as further described herein. Those of ordinary skill in the art will know of systems and techniques for incorporating a nitric oxide donor and/or a pharmaceutical agent within delivery vehicles such as a cream, gel, liquid, lotion, spray, aerosol, or transdermal patch. In some cases, the concentration of the nitric oxide donor, and/or a pharmaceutical agent in the delivery vehicle can be reduced with the inclusion of a greater amount or concentration of penetrating agent, or increased to lengthen the beneficial effect. In one set of embodiments, the nitric oxide donor and/or the pharmaceutical agent may be used in conjunction with an adjunct, such as theophylline (for example, at 10% weight by volume).

In another aspect, the present invention is directed to a kit including one or more of the compositions discussed herein. A kit typically defines a package or an assembly including one or more of the compositions of the invention, and/or other compositions associated with the invention, for example, as described herein. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein, for example, methods of promoting the making or use of compositions such as those discussed above, methods of promoting kits as discussed above, or the like. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 62/590,756, filed Nov. 27, 2017, entitled “Systems and Methods for Topical Creams for Warming,” by Gladstone, et al., is incorporated herein by reference in its entirety.

The following examples are intended to illustrate certain embodiments of the present invention, but do not exemplify the full scope of the invention.

### Example 1

This example illustrates a composition in accordance with certain embodiments of the invention.

A first, aqueous phase was prepared in a first vessel and a second, lipid phase was prepared in a second vessel. See Table 1 for the components and their concentrations. In one example, 850 g of material was prepared.

The aqueous phase was stirred while the ingredients were added, and while heating. Similarly, the lipid phase was stirred while the ingredients were added, and while heating. Both were heated to 70+/−2° C.

Next, the lipid phase was added to the aqueous phase. Then, the combined phases were cooled to 25+/−2° C. under continuous mixing.

TABLE 1

| | Concentration |
|---|---|
| Component | (wt %) |
| Aqueous phase | |
| Distilled water | 60% |
| Sodium chloride | 4% |
| Potassium chloride | 4% |
| L-arginine HCl | 12% |
| Lipid phase | |
| Mineral oil, light | 8% |
| Caprylic/capric triglycerides | 4.35% |
| Phenoxyethanol | 0.3% |
| Gelot 64 (glycerol stearate + PEG75 stearate) | 4.1% |
| Cetyl alcohol (1-hexadecanol) | 1.6% |
| Vanillyl butyl ether | 1% |
| Menthoxypropandiol | 0.1% |
| Oleoresin of capsicum | 0.25% |
| Methylparaben | 0.2% |
| Propylparaben | 0.1% |
| Total | 100% |

### Example 2

This example illustrates another composition in accordance with certain embodiments of the invention. The same protocol as Example 1 was used, except using the components and their concentrations as shown in Table 2.

TABLE 2

| | Concentration |
|---|---|
| Component | (wt %) |
| Aqueous phase | |
| Distilled water | 62.8% |
| Sodium chloride | 4.13% |
| Potassium chloride | 4.13% |
| L-arginine HCl | 8.25% |
| Lipid phase | |
| Mineral oil, light | 8.32% |
| Caprylic/capric triglycerides | 5% |
| Phenoxyethanol | 0.3% |
| Gelot 64 (glycerol stearate + PEG75 stearate) | 4.1% |
| Cetyl alcohol (1-hexadecanol) | 1.6% |
| Vanillyl butyl ether | 0.75% |
| Oleoresin of capsicum | 0.25% |
| Menthoxypropandiol | 0.075% |
| Methylparaben | 0.2% |
| Propylparaben | 0.1% |
| Total | 100% |

The mixture emulsified easily and proved to be very stable. It was easy to rub into the skin of the desired area. The cream was applied to the hands for testing purposes. Some results using this cream are discussed below.

### Example 3

A woman aged 60 with complaints of cold hands was given a sample of warm cream. She applied approximately 1.5 gm to her hands and rubbed the cream into the skin of the entire hand. After 4 minutes she noticed the warming effect which continued to increase over the course of about 20 minutes. The skin appeared slightly flushed. The warming effect lasted for several hours. The woman said she liked the moisturizing effect of the cream.

### Example 4

A male aged 65 applied approximately 1.5 gm of warm cream to his hands. The cream was rubbed into the skin of his entire hands. The warming effect was evident after a few minutes and the coloration of his hands became slightly reddened. The warming increased over the course of about 20 minutes and began to wane after about 3 hours. The effect remained evident for about 4 hours. The man said the cream had a moisturizing effect.

### Example 5

A woman aged 64 with mild complaints of cold hands applied about 1.5 gm of the warming cream to her hands. The cream was rubbed for about 3 minutes. The warming effect was evident during the rubbing process and lasted several hours.

### Example 6

This example illustrates a composition in yet another embodiment of the invention.

TABLE 3

| | Concentration |
|---|---|
| Component | (wt %) |
| Distilled water | 60.167% |
| L-arginine HCl | 11% |
| Mineral oil light | 8.02% |
| Labrafac CC (caprylic/capric triglycerides) | 4.8% |
| Sodium chloride | 4.125% |
| Potassium chloride | 4.125% |
| Gelot 64 CG (glycerol stearate + | 4.1% |
| PEG75 stearate) | |
| Cetyl alcohol (1-hexadecanol) | 1.6% |
| Xanthan gum | 0.4% |
| Isopropyl myristate | 0.4% |
| Vanillyl butyl ether | 0.375% |
| Phenoxyethanol | 0.3% |
| Oleoresin of capsicum | 0.25% |
| Methylparaben | 0.2% |
| Propylparaben | 0.1% |
| Menthoxypropandiol | 0.0375% |

### Example 7

This example illustrates a composition in still another embodiment of the invention.

TABLE 4

| | Concentration |
|---|---|
| Component | (wt %) |
| Distilled water | 59.75% |
| L-arginine HCl | 11% |
| Mineral oil Light | 8.02% |
| Labrafac CC (caprylic/capric triglycerides) | 4.8% |
| Sodium chloride | 4.125% |
| Potassium chloride | 4.125% |
| Gelot 64 CG (glycerol stearate + | 4.1% |
| PEG75 stearate) | |
| Cetyl alcohol (1-hexadecanol) | 1.6% |
| Vanillyl butyl ether | 0.75% |
| Xanthan gum | 0.4% |
| Isopropyl myristate | 0.4% |
| Phenoxyethanol | 0.3% |
| Oleoresin of capsicum | 0.25% |
| Methylparaben | 0.2% |
| Propylparaben | 0.1% |
| Menthoxypropandiol | 0.075% |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A topical delivery vehicle for application to the skin of a subject, the topical delivery vehicle comprising:
L-arginine and/or an L-arginine salt;
one or more ionic salts at a concentration of at least 5 wt %;
oleoresin of capsicum;
a warming agent having an onset time of less than 1 minute; and
a cooling agent,
wherein the topical delivery vehicle is a cream.

2. The topical delivery vehicle of claim 1, wherein the topical delivery vehicle comprises L-arginine.

3. The topical delivery vehicle of any one of claim 1 or 2, wherein the topical delivery vehicle comprises L-arginine HCl.

4. The topical delivery vehicle of any one of claims 1-3, wherein the topical delivery vehicle comprises L-arginine and/or an L-arginine salt at a concentration of at least 5 wt %.

5. The topical delivery vehicle of any one of claims 1-4, wherein the topical delivery vehicle comprises L-arginine and/or an L-arginine salt at a concentration of at least 10 wt %.

6. The topical delivery vehicle of any one of claims 1-5, wherein the topical delivery vehicle comprises sodium chloride.

7. The topical delivery vehicle of any one of claims 1-6, wherein the topical delivery vehicle comprises potassium chloride.

8. The topical delivery vehicle of any one of claims 1-7, wherein the ionic salt is present at a concentration of at least 7 wt %.

9. The topical delivery vehicle of any one of claims 1-8, wherein the ionic salt is present at a concentration of at least 10 wt %.

10. The topical delivery vehicle of any one of claims 1-9, wherein the oleoresin of capsicum is present at a concentration of at least 0.01 wt %.

11. The topical delivery vehicle of any one of claims 1-10, wherein the oleoresin of capsicum is present at a concentration of between 0.01 wt % and 0.1 wt %.

12. The topical delivery vehicle of any one of claims 1-11, wherein the cooling agent is present at a concentration of between 0.01 wt % and 0.1 wt %.

13. The topical delivery vehicle of any one of claims 1-12, wherein the warming agent comprises vanillyl butyl ether.

14. The topical delivery vehicle of any one of claims 1-13, wherein the warming agent is present at a concentration of at least 0.5 wt %.

15. The topical delivery vehicle of any one of claims 1-14, wherein the warming agent is present at a concentration of between 0.5 wt % and 1.5 wt %.

16. The topical delivery vehicle of any one of claims 1-15, wherein the cooling agent comprises menthoxypropandiol.

17. The topical delivery vehicle of any one of claims 1-16, wherein the cooling agent is present at a concentration of at least 0.05 wt %.

18. The topical delivery vehicle of any one of claims 1-17, wherein the cooling agent is present at a concentration of between 0.05 wt % and 0.5 wt %.

19. The topical delivery vehicle of any one of claims 1-18, wherein the subject is human.

20. A method, comprising applying the composition of any one of claims 1-19 to a subject.

21. A topical delivery vehicle for application to the skin of a subject, the topical delivery vehicle comprising:
a nitric oxide donor;
one or more capsaicin compounds;
a warming agent having an onset time of less than 1 minute; and
a cooling agent,
wherein the topical delivery vehicle has an ionic strength of at least 0.25 M, and is a cream, gel, lotion, or ointment.

22. The topical delivery vehicle of claim 21, wherein the nitric oxide donor comprises L-arginine.

23. The topical delivery vehicle of any one of claim 21 or 22, wherein the nitric oxide donor comprises L-arginine HCl.

24. The topical delivery vehicle of any one of claims 21-23, wherein the nitric oxide donor is present at a concentration of at least 5 wt %.

25. The topical delivery vehicle of any one of claims 21-24, wherein the nitric oxide donor is present at a concentration of at least 7 wt %.

26. The topical delivery vehicle of any one of claims 21-25, wherein the nitric oxide donor is present at a concentration of at least 10 wt %.

27. The topical delivery vehicle of any one of claims 21-26, wherein the composition comprises oleoresin of capsicum.

28. The topical delivery vehicle of any one of claims 21-27, wherein the one or more capsaicin compounds are present at a concentration of at least 0.01 wt %.

29. The topical delivery vehicle of any one of claims 21-28, wherein the one or more capsaicin compounds are present at a concentration of at least 0.01 wt % and 0.1 wt %.

30. The topical delivery vehicle of any one of claims 21-29, wherein the warming agent comprises vanillyl butyl ether.

31. The topical delivery vehicle of any one of claims 21-30, wherein the warming agent is present at a concentration of at least 0.5 wt %.

32. The topical delivery vehicle of any one of claims 21-31, wherein the warming agent is present at a concentration of between 0.5 wt % and 1.5 wt %.

33. The topical delivery vehicle of any one of claims 21-32, wherein the cooling agent comprises menthoxypropandiol.

34. The topical delivery vehicle of any one of claims 21-33, wherein the cooling agent is present at a concentration of at least 0.05 wt %.

35. The topical delivery vehicle of any one of claims 21-34, wherein the cooling agent is present at a concentration of between 0.05 wt % and 0.5 wt %.

36. The topical delivery vehicle of any one of claims 21-35, wherein the topical delivery vehicle has an ionic strength of at least 0.5 M.

37. The topical delivery vehicle of any one of claims 21-36, wherein the topical delivery vehicle has an ionic strength of at least 1 M.

38. The topical delivery vehicle of any one of claims 21-37, wherein the topical delivery vehicle comprises sodium chloride.

39. The topical delivery vehicle of any one of claims 21-38, wherein the topical delivery vehicle comprises potassium chloride.

40. The topical delivery vehicle of any one of claims 21-39, wherein the composition is a cream.

41. The topical delivery vehicle of any one of claims 21-40, wherein the composition is a gel.

42. The topical delivery vehicle of any one of claims 21-41, wherein the composition is a lotion.

43. The topical delivery vehicle of any one of claims 21-42, wherein the composition is an ointment.

44. The topical delivery vehicle of any one of claims 21-43, wherein the subject is human.

45. A method, comprising applying the composition of any one of claims 21-44 to a subject.

46. A topical delivery vehicle for application to the skin of a subject, wherein at least about 80% by weight of the topical delivery vehicle comprises:
water;
sodium chloride and/or potassium chloride;
L-arginine and/or L-arginine HCl;
oleoresin of capsicum;
vanillyl butyl ether;
menthoxypropandiol; and
an oil.

47. A topical delivery vehicle for application to the skin of a subject, the topical delivery vehicle being a cream and comprising:
water;
one or more ionic salts;
one or more nitric oxide donors;
oleoresin of capsicum;
vanillyl butyl ether;
menthoxypropandiol;
an oil;
one or more triglycerides;
phenoxyethanol;
one or more stearates;
cetyl alcohol; and
methylparaben and/or propylparaben.

48. A topical delivery vehicle for application to the skin of a subject, the topical delivery consisting essentially of:
water;
one or more ionic salts;
one or more nitric oxide donors;
oleoresin of capsicum;
vanillyl butyl ether;
menthoxypropandiol;
an oil;
one or more triglycerides;
phenoxyethanol;
one or more stearates;
cetyl alcohol; and
methylparaben and/or propylparaben.

49. A topical delivery vehicle for application to the skin of a subject, the topical delivery vehicle comprising each of the following compounds at concentrations that differ no more than +/−20% of the stated concentrations:
water at a concentration of 30% to 75% by weight;
sodium chloride and/or potassium chloride at a concentration of 5% to 15% by weight;
L-arginine and/or L-arginine HCl at a concentration of 5% to 15% by weight;
oleoresin of capsicum at a concentration of 0.1% to 5% by weight;
vanillyl butyl ether at a concentration of 0.1% to 5% by weight;
menthoxypropandiol at a concentration of 0.1% to 5% by weight;
mineral oil at a concentration of 5% to 20% by weight;
one or more triglycerides at a concentration of 1% to 10% by weight;
one or more stearates at a concentration of 1% to 10% by weight;
phenoxyethanol at a concentration of 0.1% to 5% by weight; and
methylparaben and/or propylparaben at a concentration of 0.1% to 5% by weight.
